(19) United States
(12) Patent Application Publication
(10) Pub. No.: US 2017/0313808 A1
(43) Pub. Date: Nov. 2, 2017

(54) BIOACTIVE POLYMERIC LIQUID FORMULATIONS OF ABSORBABLE, SEGMENTED APLIPHATIC POLYURETHANE COMPOSITIONS

(71) Applicants: Shalaby W. Shalaby, Anderson, SC (US); Joel T. Corbett, Anderson, SC (US); Michael Aaron Vaughn, Anderson, SC (US); David Ingram, Anderson, SC (US)

(72) Inventors: Shalaby W. Shalaby, Anderson, SC (US); Joel T. Corbett, Anderson, SC (US); Michael Aaron Vaughn, Anderson, SC (US); David Ingram, Anderson, SC (US)

(21) Appl. No.: 15/652,135

(22) Filed: Jul. 17, 2017

Related U.S. Application Data
(63) Continuation-in-part of application No. 14/714,699, filed on May 18, 2015.
Application No. 14/714,699 is a continuation of application No. 14/058,640, filed on Oct. 21, 2013, now Pat. No. 9,034,361.
Application No. 14/058,640 is a continuation of application No. 13/415,415, filed on Mar. 8, 2012, now abandoned.
Application No. 13/415,415 is a continuation of application No. 12/380,391, filed on Feb. 26, 2009, now abandoned.
Application No. 12/380,391 is a continuation-in-part of application No. 14/618,531, filed on Feb. 10, 2015.
Application No. 14/618,531 is a continuation of application No. 12/454,774, filed on May 22, 2009, now Pat. No. 8,952,075.
(60) Provisional application No. 61/069,046, filed on Mar. 12, 2008.
(60) Provisional application No. 61/128,487, filed on May 22, 2008.

(51) Int. Cl. C08G 18/73; C08G 18/42; A61L 27/18; C08G 64/02; C08G 18/48; A61L 27/52; A61L 24/00; C08G 18/34; C08G 18/32; C08G 18/12; C08L 75/04; C08G 18/44

(52) U.S. Cl. CPC C08G 18/73; C08L 75/04; C08G 18/4244; A61L 27/18; C08G 64/0241; C08G 18/4887; C08G 18/44; C08G 18/4283; C08G 18/428; A61L 24/0021; C08G 18/341; C08G 18/3206; C08G 18/12; A61L 27/52; A61L 2430/06; C08G 2230/00

(57) ABSTRACT

Bioactive liquid formulations are formed of combinations of absorbable, segmented aliphatic polyurethane compositions and liquid polyether for use as vehicles for the controlled release of at least one active agent for the conventional and unconventional treatment of different forms of cancer and the management of at least one type of bacterial, fungal, and viral infection.

### CROSS-REFERENCE TO RELATED APPLICATIONS

This application is a continuation-in-part of U.S. patent application Ser. No. 14/714,699, filed May 18, 2015, which is a continuation of U.S. patent application Ser. No. 14/058,640, filed Oct. 21, 2013, now U.S. Pat. No. 9,034,361, issued on May 19, 2015, which is a continuation of U.S. application Ser. No. 13/415,415, filed Mar. 8, 2012, which is a continuation of U.S. patent application Ser. No. 12/380,391, filed Feb. 26, 2009, which claims the benefit under 35 U.S.C. §119(e) of U.S. Provisional Patent Application No. 61/069,046, filed Mar. 12, 2008, and this application is a continuation-in-part of U.S. patent application Ser. No. 14/618,531, filed Feb. 10, 2015, which is a continuation of U.S. patent application Ser. No. 12/454,774, filed May 22, 2009, now U.S. Pat. No. 8,952,075, issued on Feb. 10, 2015, which claims the benefit under 35 U.S.C. §119(e) of U.S. Provisional Patent Application No. 61/128,487, filed May 22, 2008, where all the aforementioned applications are incorporated herein by reference in their entireties for all purposes.

### FIELD OF THE INVENTION

This invention is directed to bioactive polymeric liquid formulations, for example, of absorbable, segmented aliphatic polyurethane compositions which are formed of macromolecular polyether-carbonate-urethane, polyether-carbonate-urethane-urea, and polyether-ester-urethane chains in combination with liquid polyethers for use as controlled release vehicles for at least one drug capable of exhibiting at least one function associated with antibacterial, antifungal, antiviral, and/or antineoplastic activity.

### BACKGROUND OF THE INVENTION

Polyurethanes represent a main class of synthetic elastomers applied for long-term, medical implants as they present tunable chemical properties, excellent mechanical properties, good blood compatibility, and also can be designed to degrade in biological environments (A. Rechichi et al., J. Biomed. Mater. Res., 84-A, 847 (2008)). More specifically, polyether-urethane (PEU) and polyether-urethane-urea (PEUU) elastomers have long been considered ideal for use in many implanted devices, in spite of occasionally cited drawbacks (M. A. Schubert et al., J. Biomed. Mater. Res., 35, 319 (1997); B. Ward et al., J. Biomed. Mater. Res., 77-A, 380 (2008)). Of the cited drawbacks are those associated with (1) the generation of aromatic diamines, which are considered to be toxic upon degradation of segmented copolymers made using aromatic diisocyanates for interlinking; (2) chain degradation due to oxidation or radiation degradation of the polyether component of segmented copolymers, and particularly those which encounter frequent mechanical stresses in the biological environment; and (3) chemical degradation in chemically and mechanically hostile biological environments of the urethane links of segmented copolymers and particularly those comprising reactive aromatic urethane linkages.

Liquid solventless, complex polymeric compositions, which thermoset at ambient temperatures through additional polymerization of a two-component system, wherein the first component comprises amine or acrylate-terminated polyurethanes or polyurethane-ureas and the second component comprises di- or polyacrylates have been described in U.S. Pat. No. 4,742,147. However, the prior art is virtually silent on self-standing PEU and PEUU liquid solventless compositions for use in pharmaceutical formulations and/or medical devices. Similarly, the prior art on polyether-urethanes is practically silent on hydroswellable (or water-swellable) systems, in spite of the fact that it covered elastomeric, segmented, hydrophilic polyether-urethane-based, lubricious coating compositions based on aromatic diisocyanate and polyethylene glycol (U.S. Pat. No. 4,990,357)—it did not suggest a self-standing material for medical device applications.

Collective analysis of the prior art on PEU and PEUU as discussed above regarding the drawbacks of the disclosed systems, absence of self-standing liquid and hydroswellable copolymers, and recognition of the need for new materials exhibiting properties that cannot be met by those of the prior art, provided a strong incentive to explore the synthesis and evaluation of the PEU and PEUU systems subject of this invention, which are structurally tailored for their effective use in existing and new applications.

Advanced developments in the area of absorbable polymers and particularly those dealing with liquids and hydrogel-forming liquids made of copolyester and polyether-esters, respectively, were paralleled by a similarly advanced development of controlled drug delivery systems by the present inventor and coworkers for use as extrudable or injectable liquid formulations for use in parenteral and topical applications (U.S. Pat. Nos. 5,653,992; 5,714,159; 6,413,539). Pertinent to the present invention are the injectable hydrogel-forming, self-solvating, liquid, absorbable, segmented polyether-esters, which are used, in part, for the controlled release of antibacterial agents, such as doxycycline, for the treatment of periodontitis (U.S. Pat. Nos. 5,714,159; 6,413,539). The main attributes of hydrogel-forming liquid polyether-esters include their ease of application topically and as an injectable formulation without the need of using an organic solvent.

However, in spite of the extensive development and use of segmented polyurethanes for biomedical application, the prior art was silent on the development of absorbable, segmented polyurethane compositions, which can be used independently or as part of a polymeric liquid formulation for the controlled release of a broad range of bioactive agents for use in topical, parenteral, and/or injectable applications.

The present invention deals, in part, with hydroswellable, absorbable, aliphatic segmented polyurethanes and polyurethane-ureas capable of swelling in the biological environment. Attributes associated with these polymers and the technological and clinical success of the hydrogel-forming liquid polyether-esters discussed above, provided the incentive to pursue the study associated with the present invention.

### SUMMARY OF THE INVENTION

In one aspect, the present invention is directed to a bioactive polymeric liquid formulation comprising a blend of an absorbable, segmented, aliphatic polyurethane, a liquid polyoxyalkylene and at least one drug selected from doxycycline, mitomycin, clindamycin, miconazole, clotrimazole, ketoconazole, fluconazole, butoconazole, tioconazole, leflunomide, 5-fluorouracil, paclitaxel, carboplatin, mycophenolic acid, podophyllinic acid, podophyllotoxin and related bioactive compounds, wherein the liquid polyoxyalkylene comprises chains of at least one type of oxyalkylene sequence selected from oxyethylene, oxypropylene, and oxytrimethylene. The polyurethane composition comprises polyoxyalkylene chains covalently linked to alkylene carbonate chains, wherein the polyalkylene carbonate chains are interlinked with aliphatic urethane segments, wherein the alkylene carbonate chains comprise trimethylene carbonate sequences and the aliphatic urethane segments are derived from at least one diisocyanate selected from the group consisting of tetramethylene diisocyanate, hexamethylene diisocyanate, lysine-derived diisocyanate, and cyclohexane bis (methylene isocyanate), and wherein the segmented polyurethane is made by the method comprising the steps of end-grafting polyethylene glycol having a molecular weight of about 400 Da with trimethylene carbonate, and interlinking the end-grafted polyethylene glycol with hexamethylene diisocyanate. And such formulation is used as (a) a vehicle for the controlled release of at least one antineoplastic agent for treating at least one type of cancer selected from breast, ovarian, cervical, lung, prostate, testicular, and skin cancer, wherein said formulation contains at least one antineoplastic agent is selected from the group consisting of paclitaxel, 5-fluorouracil, podophyllinic acid, mycophenolic acid, and carboplatin, alternatively, the said vehicle contains at least one antineoplastic agent is selected from antimicrobial agents and immunosuppressant agents selected from the group consisting of doxycycline, tetracycline, mitomycin, clindamycin, miconazole, ketoconazole, fluconazole, and leflunomide; (b) a vehicle for the controlled release of at least one antifungal agent for treating vaginal yeast, nail and skin fungal infections, the agent selected from the group consisting of miconazole, ketoconazole, butoconazole, clotrimazole; (c) a vehicle for the controlled release of at least one antibacterial agent for treating vaginal and skin bacterial infections, the agent selected from the group consisting of doxycycline, mitomycin, and clindamycin; (d) a vehicle for the controlled release of at least one antibacterial agent for treating periodontitis and related dental infections, the agent selected from the group consisting of doxycycline, tetracycline, clindamycin, and mitomycin; and (e) a vehicle for the controlled release of at least one agent for treating genital, nail, and skin warts, and related infections, the agent selected from the group consisting of paclitaxel, 5-fluorourecil, podophyllinic acid, podophyllotoxin, miconazole, ketoconazole, butoconazole, fluconazole, and clotrimazole.

A key aspect of this invention deals with a bioactive polymeric liquid formulation which is a blend of an absorbable, segmented, aliphatic polyurethane, a liquid polyoxyalkylene and at least one drug selected from doxycycline, mitomycin, clindamycin, miconazole, clotrimazole, ketoconazole, fluconazole, butoconazole, tioconazole, leflunomide, 5-fluorouracil, paclitaxel, carboplatin, mycophenolic acid, podophyllinic acid, podophyllotoxin and related bioactive compounds, wherein the polyurethane composition comprises an aliphatic polyurethane-urea comprising polyoxyalkylene chains covalently linked to polyalkylene-urethane chains wherein the polyalkylene-urethane chains are further interlinked with aliphatic urea chain segments, and wherein the polyoxyalkylene chains comprise at least one type of oxyalkylene sequence selected from the group consisting of oxyethylene, oxypropylene, and oxytrimethylene and the urethane chain segments are derived from at least one diisocyanate selected from the group consisting of hexamethylene diisocyanate, lysine-derived diisocyanate, and cyclohexane bis (methylene isocyanate), and wherein the resulting polyoxyalkylene urethane molecules having at least one isocyanate terminal group are chain extended with an alkylene diamine selected from the group consisting of ethylene-, trimethylene-, and hexamethylene-diamine, thereby forming polyurethane-urea segmented chains. And such formulation is used as a vehicle for the controlled release of at least one antineoplastic agent for treating at least one type of cancer selected from breast, ovarian, cervical, lung, prostate, testicular, and skin cancer, wherein the at least one antineoplastic agent is selected from the group consisting of paclitaxel, 5-fluorouracil, podophyllinic acid, mycophenolic acid, and carboplatin. Alternatively, the vehicle contains at least one antineoplastic agent is selected from antimicrobial agents and immunosuppressant agents selected from the group consisting of doxycycline, tetracycline, mitomycin, clindamycin, miconazole, ketoconazole, fluconazole, and leflunomide.

Another key aspect of this invention deals with a bioactive polymeric liquid formulation which is a blend of an absorbable, segmented, aliphatic polyurethane, a liquid polyoxyalkylene and at least one drug selected from doxycycline, mitomycin, clindamycin, miconazole, clotrimazole, ketoconazole, fluconazole, butoconazole, tioconazole, leflunomide, 5-fluorouracil, paclitaxel, carboplatin, mycophenolic acid, podophyllinic acid, podophyllotoxin and related bioactive compounds, wherein the polyurethane composition comprises a polyether-ester-urethane comprising polyoxyalkylene chains covalently linked to polyester chain segments, wherein the polyester chains are interlinked with aliphatic urethane segments, wherein the polyester chain segments comprise polyester-carbonate chain segments, and further wherein the polyester chain segments are derived from at least one cyclic monomer selected from the group consisting of .epsilon.-caprolactone, p-dioxanone, 1,5-dioxepan-2-one, trimethylene carbonate, 1-lactide, glycolide, dl-lactide, and a morpholinedione. Such formulation is used (a) as a vehicle for the controlled release of at least one antineoplastic agent for treating at least one type of cancer selected from breast, ovarian, cervical, lung, prostate, testicular, and skin cancer, wherein the at least one antineoplastic agent is selected from the group consisting of antineoplastic agents consisting of paclitaxel, 5-fluorouracil, podophyllinic acid, mycophenolic acid, and carboplatin, and alternatively, the said vehicle contains at least one antineoplastic agent is selected from antimicrobial agents and immunosuppressant agents selected from the group consisting of doxycycline, tetracycline, mitomycin, clindamycin, miconazole, ketoconazole, fluconazole, and leflunomide; (b) as a vehicle for the controlled release of at least one antibacterial agent for treating periodontitis and related dental infections, the agent selected from the group consisting of doxycycline, tetracycline, clindamycin, and mitomycin; and (c) as a vehicle for the controlled release of at least one agent for treating fungus-infected nails and genital and skin warts, and related infections, the agent selected from the group consisting of paclitaxel, 5-fluorourecil, podophyllinic acid, podophyllotoxin, miconazole, ketoconazole, butoconazole, fluconazole, and clotrimazole.

Another specific aspect of the invention describes a hydroswellable, segmented, aliphatic polyurethane comprising polyoxyalkylene chains, covalently linked to polyalkylene carbonate chains, which are interlinked with aliphatic urethane segments, the composition exhibiting an at least 3 percent increase in volume when placed in the biological environment, wherein the polyoxyalkylene glycol chains comprise at least one type of oxyalkylene sequences selected from the group represented by oxyethylene, oxypropylene, oxytrimethylene, and oxytetramethylene repeat units and the alkylene carbonate chains are trimethylene carbonate sequences, and wherein the urethane segments are derived from at least one diisocyanate selected from the group represented by tetramethylene diisocyanate, hexamethylene diisocyanate, octamethylene diisocyanate, decamethylene diisocyanate, dodecamethylene diisocyanate, lysine-derived diisocyanate, and cyclohexane bis-(methylene isocyanate). Meanwhile, the polyurethane is made by reacting a liquid polyoxylene alkylene glycol comprising oxyethylene or a combination of oxyethylene and oxypropylene sequences that are end-grafted with trimethylene carbonate wherein the resulting product is interlinked with 1,6-hexane diisocyanate, and wherein the liquid polyalkylene glycol is a polyethylene glycol having, preferably, a molecular weight of about 400-500 Da. From a pharmaceutical application perspective, the polyurethanes can be used as vehicles for a controlled release formulation of at least one bioactive agent selected from the group of agents known to exhibit anti-inflammatory, anesthetic, cell growth promoting, antimicrobial, antiviral, and antineoplastic activities. In a specific pharmaceutical application, the controlled release formulation comprises at least one antimicrobial agent after treating periodontitis or bone infection selected from the group represented by doxycycline, gentamicin, vancomycin, tobramycin, clindamycin, and mitomycin and the periodontal formulation may include absorbable microparticles made of acid-terminated glycolide-based polyester and a liquid excipient such as a liquid polyethylene glycol and an alkylated or acylated derivative thereof. In a second group of pharmaceutical applications, the controlled release formulation comprises a liquid polyethylene glycol or an alkylated or acylated derivative thereof as an excipient and at least one bioactive agent selected from the group represented by paclitaxel, carboplatin, miconazole, leflunamide, ciprofloxacin, and a recombinant protein for treating breast or ovarian cancer in humans or animals. Additionally, for tissue repair applications, the polyurethane can be admixed with one or more cyanoacrylate monomer for use as a rheological modifier of tissue adhesives, wherein the one or more cyanoacrylate monomer is part of an absorbable or non-absorbable tissue adhesive formulation comprising stabilizers against premature polymerization, free radically and anionically, and at least one monomer selected from the group represented by ethyl-, butyl-, isobutyl-, methoxypropyl-, methoxyethyl-, and methoxybutyl cyanoacrylate.

Another specific aspect of the present invention deals with a hydroswellable, segmented, aliphatic polyurethane-urea comprising polyoxyalkylene chains covalently interlinked with polyalkylene urethane segments, which are further interlinked with aliphatic urea chain segments, the composition exhibiting at least 5 percent increase in volume when placed in the biological environment, wherein the polyalkylene glycol chains comprise at least one type of oxyalkylene sequences selected from the group represented by oxyethylene, oxypropylene, oxytrimethylene, and oxytetramethylene repeat units and the urethane segments are derived from at least one diisocyanate selected from the group represented by hexamethylene diisocyanate, hexamethylene diisocyanate, octamethylene diisocyanate, decamethylene diisocyanate, dodecamethylene diisocyanate, 1,4 cyclohexane diisocyanate, lysine-derived diisocyanate, and cyclohexane bis(methylene isocyanate) and wherein the resulting polyoxyalkylene urethane molecules having at least one isocyanate terminal group are chain-extended with an alkylene diamine selected from the group represented by ethylene-, trimethylene, tetramethylene-, hexamethylene-, and octamethylene-diamine, thus forming polyetherurethane-urea segmented chains.

A clinically important aspect of the invention deals with a hydroswellable, segmented, aliphatic polyurethane-urea comprising polyoxyalkylene chains covalently interlinked with polyalkylene urethane segments, which are further interlinked with aliphatic urea chain segments, the composition exhibiting at least 5 percent increase in volume when placed in the biological environment, wherein the polyurethane-urea (1) can be chemically crosslinked, wherein the crosslinking is achieved using an alkylene diisocyanate; (2) can exhibit microporosity with a practically continuous cellular structure; (3) can comprise at least one covalently bonded aromatic group to stabilize the chain against radiation and oxidation degradation; and/or (4) can be used as an artificial cartilage for restoring the function of diseased or defective articulating joints in humans and animals.

An important aspect of this invention deals with a hydroswellable, segmented, aliphatic polyurethane comprising polyoxyalkylene chains covalently linked to polyester or polyester-carbonate chain segments, interlinked with aliphatic urethane segments, the composition exhibiting at least 5 percent increase in volume when placed in the biological environment, wherein the polyester or polyester-carbonate chain segments are derived from at least one cyclic monomer selected from the group represented by E-caprolactone, trimethylene carbonate, p-dioxanone, 1,5-dioxepan-2-one, 1-lactide, dl-lactide, glycolide, and a morpholinedione. Meanwhile, the polyurethane can exhibit microporosity with practically continuous cellular structure for use as an absorbable scaffold or part thereof for cartilage tissue engineering, with or without the aid of a cell growth promoting agent therein.

For prolonged effective device performance, the present invention is also directed to a hydroswellable, segmented, aliphatic polyurethane-urea comprising a combination of linear functionalized polysiloxane and polyoxyalkylene chains interlinked with polyalkylene urethane segments, which are further interlinked with aliphatic urea chain segments, the composition exhibiting at least 5 percent increase in volume when placed in the biological environment, wherein the polyoxyalkylene chain comprises at least one type of oxyalkylene sequences selected from the group represented by oxyethylene, oxypropylene, oxytrimethylene, and oxytetramethylene repeat units and the functionalized polysiloxane is derived from bis-hydroxyalkyl-terminated polysiloxane comprising at least dimethoxysiloxane internal sequences and two hydroxyalkyl or aminoalkyl terminals and further wherein the urethane segments are derived from at least one diisocyanate selected from the group represented by hexamethylene diisocyanate, octamethylene diisocyanate, decamethylene diisocyanate, dodecamethylene diisocyanate, 1,4 cyclohexane diisocyanate, lysine-derived diisocyanate, and cyclohexane bis(methylene isocyanate) and wherein the resulting polyoxyalkylene urethane molecules having at least one isocyanate terminal group are further chain-extended with an alkylene diamine selected from the group represented by ethylene-, trimethylene, tetramethylene-, hexamethylene- and octamethylene-diamine, thus forming polyetherurethane-urea segmented chains, wherein the polyurethane-urea (1) can be chemically crosslinked wherein the crosslinking is achieved using an alkylene diisocyanate; (2) can exhibit microporosity with a practically continuous cellular structure; (3) can comprise at least one covalently bonded aromatic group to stabilize the chain against radiation and oxidation degradation; and/or (4) can be used as an artificial cartilage for restoring the function of diseased or defective articulating joints in humans and animals.

In a further aspect, the present invention is directed to hydroswellable (or water-swellable) absorbable and non-absorbable aliphatic, segmented polyurethanes and polyurethane-ureas, which can undergo swelling when placed in the biological environment manifested through an at least 3 percent increase in volume by virtue of having a highly hydrophilic polyalkylene oxide as an inherent part of their segmented chain molecules. By varying the type and fraction of the different segments constituting the copolymers, their pharmaceutical and biomedical applications as non-absorbable and absorbable materials entail their use in carriers for the controlled release of bioactive agents, rheological modifiers of absorbable and non-absorbable cyanoacrylate tissue adhesives, synthetic cartilage-like materials, and scaffolds for tissue engineering cartilage tissues.

### DETAILED DESCRIPTION OF PREFERRED EMBODIMENTS

The present invention is directed to bioactive, polymeric, liquid formulations which are absorbable, segmented, aliphatic liquid polyurethane compositions or a combination thereof with a liquid polyether. The absorbable, segmented, aliphatic polyurethane compositions comprise polyether-carbonate-urethane-urea, polyether-carbonate-urethanes, polyether-carbonate-ester-urethane, and/or polyether-ester-urethane. These polyurethane compositions, and preferably the combination with a liquid polyether to reduce their viscosities, are selected to be easily applied topically as drug-loaded formulations, which can be also extruded or injected by syringe or collapsible dispenser into the biological site. Some of these formulations, particularly those containing a water-soluble liquid polyethylene glycol, tend to undergo gelation or at least swelling upon contacting the liquid environment at the application site. The extent of swelling or gelation is used to control the drug release profile by adjusting the hydrophilicity of the polyurethane compositions and/or the fraction of the polyethylene glycol. This strategy is used to control the diffusion of the active agent and hence its release profile. As to the types of bioactive agents, subject of this invention, in terms of their intended use in a traditional and well-established manner, they fall into five main categories, namely: (1) antibacterial as in doxycycline, mitomycin, clindamycin; (2) antifungal as in miconazole, clotrimazole, tioconazole, and ketoconazole; (3) antineoplastic as in 4-fluorouracil, paclitaxel, carboplatin, mycophenolic acid, and podophyllinic acid; (4) antiviral as in podophyllotoxin; and (5) immunosuppressive as in leflunomide. A unique aspect of the bioactive agents, subject of this invention, is the newly coined category of drugs which are denoted in the instant application as crossover bioactive agents, each of which has a primary function and yet exhibits at least one additional function that is distinctly different from its primary function. Given in Table I are typical examples that are part of this invention and were not cited in the prior art or can be considered obvious to those familiar with the biochemical, physiological, and pharmacological aspects of drugs.

TABLE I
Typical Crossover Bioactive Agents

| | Primary | |
|---|---|---|
| Drug Name | Clinical Use | Additional Uses and Properties |
| Miconazole | Antifungal | Antineoplastic (as per testing |
| | | with ovarian cancer cell lines |
| | | (SKOV3 and OVCAR). |
| Fluconazole | Antifungal | Antineoplastic (as per testing |
| | | with ovarian cancer cell lines |
| | | (SKOV3 and OVCAR). |
| Mitomycin | Antibacterial | Antineoplastic, as per testing |
| | | with ovarian cancer cell lines |
| | | (SKOV3 and OVCAR). |
| Clindamycin | Antibacterial | Antineoplastic, as per testing |
| | | with ovarian cancer cell lines |
| | | (SKOV3 and OVCAR). |
| Paclitaxel | Antineoplastic | Antibacterial as per testing |
| | | with S aureous |
| 5-Fluorouracil | Antineoplastic | Antibacterial as per testing |
| | | with S aureous |
| Leflunomide | Immunosuppressant | Antineoplastic, as per testing |
| | | with ovarian cancer cell lines |
| | | (SKOV3 and OVCAR). |

From a clinical perspective, this invention provides bioactive formulations that are useful for treating bacterial, fungal, and viral infections as well as different forms of cancers. A key aspect of this invention deals with the crossover drugs having multipurpose functions as in the case of (1) miconazole and fluconazole, which are not only useful for treating yeast infections, but also exhibit antineoplastic and antiviral activities and thereby are applicable for treating several forms of cancer and treatment of human immunodeficiency virus (HIV); (2) leflunomide, which is not only an immunosuppressant, but also useful as an antineoplastic agent for treating different forms of cancer as well as an antiviral agent for managing HIV infection; and (3) mitomycin and clindamycin, which are not only antibacterial agents, but also exhibit antineoplastic and antiviral activities and thereby are useful for treating different forms of cancer and managing HIV infection, respectively; and (4) paclitaxel and 5-fluorouracil, which are not only antineoplastic agents, but are also useful for treating bacterial infections and managing HIV infection.

For the preparation of certain bioactive formulations, there may be (1) no need to use the liquid polyether as in the case of the polyurethane composition, which is sufficiently flowable and its viscosity allows the final formulation to be injectable through a syringe or extrudable through a squeezable dispenser; (2) a need to use microparticular anion-exchangers made of carboxyl-terminated polyglycolide similar to that described in U.S. Pat. Nos. 5,714,159 and 6,413,539 should the active agent be basic and can interact ionically with the anion-exchanger thereby modulating its release profile; and (3) a need to prepare a low viscosity diluent polyurethane composition having a high polyether content, but having qualitatively the same component as the polyurethane composition used as the main vehicle.

In addition, the present invention is generally directed to the tailored synthesis of the following families of hydroswellable polymers. The term “hydroswellable” is intended to indicate that the polymers swell and increase in volume in the presence of water.

(1) Relatively slow-absorbing. segmented polyether-carbonate-urethanes (PECU) as vehicles for the controlled release of bioactive agents including those known to exhibit or unexpectedly exhibit antimicrobial, microbicidal, antineoplastic, and antiviral activities wherein the typical PECUs (a) exhibit <20 percent or no solubility in water, (b) are made to be liquids at about 50° C.; (c) have a weight average molecular weight exceeding 10 kDa; (d) swell in an aqueous environment leading to an increase of volume of at least 3 percent; and (e) are miscible in water-soluble, low viscosity liquid excipients, such as polyethylene glycol 400, to facilitate their use as injectable formulations that undergo gel-formation when introduced to aqueous biological sites—the ratio of the PECU to the excipient can be modulated in concert with the active agent solubility, its intended release site, and preferred release rate.

(2) The PECUs of Item 1 as rheology modifiers of cyanoacrylate-based tissue adhesive formulations wherein (a) the PECU is used to increase the viscosity of the uncured tissue adhesive; (b) render the cured tissue adhesive more compliant and able to conform with the biological site—this is achieved by decreasing the cured adhesive modulus due to the presence of the low modulus PECU at concentrations of at least one weight percent; (c) the cyanoacrylate tissue adhesive comprises at least one monomer selected from the group represented by ethyl-, n-butyl-, isobutyl-, methoxypropyl-, ethoxypropyl-, methoxybutyl-, and octyl-cyanoacrylate; and (d) the cyanoacrylate tissue adhesive contains at least one stabilizer to prevent premature polymerization by an anionic and free radical mechanism-typical examples of these are pyrophosphoric acid and butylated hydroxyl anisole for stabilization against anionic and free radical polymerization, respectively.

(3) Relatively fast-absorbing, segmented aliphatic polyether-ester urethanes (PEEU) and polyether-carbonate-ester urethanes (PECEU) as vehicles for the controlled release of bioactive agents including those known to exhibit or unexpectedly exhibit antimicrobial, microbicidal, antiviral, and antineoplastic activities wherein the typical PEEUs and PECEUs (a) exhibit limited (<20 percent) or no solubility in water; (b) are made to be liquids at about 50° C.; (c) have a weight average molecular weight exceeding 10 kDa; (d) swell in an aqueous environment leading to an increase of volume of at least 3 percent; and (e) are miscible in water-soluble, low viscosity liquid excipients, such as polyethylene glycol 400 and an alkylated or acylated derivative thereof, to facilitate their use as injectable formulations that undergo gel-formation when introduced to aqueous biological sites—the ratio of the PECU to the excipient can be modulated in concert with the active agent solubility, its intended release site, and preferred release rate.

(4) The PEEUs and PECEUs of Item 3 as rheology modifiers of absorbable cyanoacrvlate-based tissue adhesive formulations wherein (a) the PEEU or PECEU is used to increase the viscosity of the uncured tissue adhesive; (b) render the cured tissue adhesive more compliant and able to conform with the biological site—this is achieved by decreasing the cured adhesive modulus due to the presence of the low modulus PEEU or PECEU at concentrations of at least one weight percent; (c) the cyanoacrylate tissue adhesive comprises an alkoxyalkyl cyanoacrylate, such as methoxypropyl cyanoacrylate or a mixture of an alkoxyalkyl cyanoacrylate and an alkyl cyanoacrylate, such as ethyl cyanoacrylate; and (d) the cyanoacrylate tissue adhesive contains at least one stabilizer to prevent premature polymerization by an anionic and free radical mechanism—typical examples of these are pyrophosphoric acid and butylated hydroxyl anisole for stabilization against anionic and free radical polymerization, respectively.

(5) Essentially biostable, non-absorbable, segmented, aliphatic polyether urethane-ureas (PEUU) as flexible, solid, linear or chemically crosslinked polymers for use primarily as cartilage-like materials, which undergo swelling and deswelling upon cyclic application of compressive force for prolonged periods, while practically maintaining their initial properties, wherein the typical PEUUs (a) exhibit limited (<5 percent) or no solubility in water; (b) can be fabricated into films, sheets or caps for articulating bones in humans or animals with essentially no display of first order thermal transitions and exhibiting ultimate elongation exceeding 200 percent, reversible elongation of >10 percent and an at least 5 percent increase in volume when immersed in water for less than two hours; (c) have a molecular weight corresponding to an inherent viscosity of more than unity using hexafluoro-isopropyl alcohol (HFIP) as a solvent when present as linear molecular chains; and (d) can be fabricated into different desirable forms or geometries by solution casting.

(6) Highly biostable, non-absorbable, segmented, aliphatic PEUU as in Item 5 comprising a polysiloxane (e.g., poly dimethyl siloxane segment) to improve its oxidation stability in the biological environment.

(7) Highly biostable, non-absorbable, segmented, aliphatic PEUU as in Item 5 comprising a covalently bonded chemical entity capable of minimizing or eliminating radiation during radiation sterilization, and oxidative degradation when placed in the biological environment. These radiation and oxidation stabilizers can be in the form of polymerizable (as in diols) derivatives of hydroxyl aromatic compounds or low molecular polymers comprising oxy-aromatic groups and hydroxyl end-groups. Such simple or polymeric diols can be mixed with the polyether diol prior to end-grafting with other monomers and interlinking with diisocyanate.

(8) Absorbable, segmented, aliphatic polyether-ester urethane (APEEU) and polyether-ester-carbonate urethane (APEECU) as scaffolds for cartilage tissue engineering wherein the typical APEEUs and APEECUs (a) comprise polyoxyalkylene chains (such as those derived from polyethylene glycol and block or random copolymers of ethylene oxide and propylene oxide) covalently linked to polyester or polyester-carbonate segments (derived from at least one monomer selected from the group represented by trimethylene carbonate, ε-caprolactone, lactide, glycolide, p-dioxanone, 1,5-dioxepan-2-one, and a morpholinedione) and interlinked with aliphatic urethane segments derived from 1,6 hexamethylene-, 1-4 cyclohexane-, cyclohexane-bis-methylene-, 1,8 octamethylene- or lysine-derived diisocyanate; (b) display at least 5 percent increase in volume due to swelling, when placed in the biological environment; (c) have a microporous structure with average pore size ranging between about 20 and 400 micron and practically continuous cell structure; and (d) are suitable for use as an absorbable scaffold for cartilage tissue engineering wherein the scaffold may contain at least one bioactive agent which may include at least one cell growth promoter.

From a clinical perspective, compositions and formulations or devices thereof subject of the present invention can be used in a broad-range of applications including (1) injectable gel-forming liquid formulations for the controlled delivery of bioactive agents for treating periodontitis, nail infection, bone infection, a variety of bacterial and fungal infections, and different forms of cancers; (2) in situ-forming, extrudable luminal liner for the controlled drug delivery at the luminal wall of vaginal canals and blood vessels; (3) a rheology modifier for essentially non-absorbable and absorbable cyanoacrylate-based tissue adhesive formulations; (4) cartilage-like covers to protect defective or diseased articulating joints; and (5) an absorbable scaffold for cartilage and soft tissue engineering.

Further illustrations of the present invention are provided by the following examples:

### EXAMPLES

### Example 1

### Synthesis and Characterization of a Typical Polyether-Carbonate-Urethane, P1

For an initial charge, poly(ethylene glycol) (Mₙ=400 Da) (0.15 moles) and tin(II) 2-ethyl hexanoate (3.53×10⁻⁴ moles) was added to a 500 mL, 3-neck, round-bottom flask equipped with a PTFE coated magnetic stir bar. The contents were heated to 70° C. and allowed to stir for 10 minutes. For a second charge, trimethylene carbonate (0.882 moles) was added and the contents were heated to 135° C. Conditions were maintained until practically complete monomer conversion was achieved. The magnetic stir bar was removed and replaced by a stainless steel mechanical stirrer. The polymer was cooled to room temperature. For a third charge, 1,6-diisocyanatohexane (0.12 moles) was added and the contents were stirred until complete mixing was achieved. The contents were stirred and heated to 100° C. Conditions were maintained for 1.25 hours. The polymer was allowed to cool to room temperature and then dissolved in an equal part of tetrahydrofuran. The polymer solution was treated with 5 mL of 2-propanol at 55° C. then precipitated in cold water. The purified polymer was isolated and dried to a constant weight at 55° C. on a rotary evaporator. The purified polymer was characterized for molecular weight by GPC using tetrahydrofuran as the mobile phase which resulted in an Mₙ, M_{W}, Mₚ, and PDI of 11 kDa, 19 kDa, 18 kDa, and 1.7 respectively. Identity and composition were confirmed by FT-IR and NMR, respectively.

### Example 2

### Preparation and Evaluation of Doxycycline-Containing Formulation F1-A

### Using the Polyurethane Composition P1 of Example 1

The polyurethane composition of Example 1 (4.0 g) was heated to 50° C. and mixed thoroughly with polyethylene glycol having a molecular weight of 400 Da (6.0 g). To this (without additional heating) was added a mixture of doxycycline hydrochloride (1.5 g) and microparticles of carboxyl-terminated polyglycolide (0.75 g) having an average diameter of <10 micron (prepared as described in U.S. Pat. Nos. 5,714,159 and 6,413,539). All components were mechanically mixed at room temperature until a uniform dispersion is obtained (as determined microscopically). The flow property of the formulation is measured in terms of complex viscosity using a parallel plate rheometer.

To determine the release profile of doxycycline, aliquots of the formulation F1-A were incubated in a buffered solution at 37° C. and 7.2 pH for predetermined periods of time. At the conclusion of each period, the buffer solution is decanted and replaced by a fresh aliquot.

The decanted buffer was analyzed by HPLC to determine the amount of doxycycline released. Over a period of 600 hours, about 25, 30, 35, 45, and 55 percent of the days was released at 50, 100, 200, 300, and 600 hours respectively, was released.

### Example 3

### Preparation and Evaluation of Doxycycline-Containing Formulation F1-B

### Using the Polyurethane/Composition of P1 of Example 1

This was conducted as described in Example 2 with the exception of using different amounts of P1 (5 g) and PEG-400 (5 g). Results of the drug release indicated about 22, 32, 38, 43, and 57 percent of the drug released at 50, 100, 200, 300, and 600 hours, respectively.

### Example 4

### Synthesis and Characterization of Polyether-Ester-Urethane: General Method

For an initial charge, poly(ethylene glycol) (Mₙ=400 Da) and tin(II) 2-ethyl hexanoate was added to a 500 mL, 3-neck, round-bottom flask equipped with a PTFE coated magnetic stir bar. The contents were heated to 70° C. and allowed to stir for 10 minutes. For a second charge, dl-lactide and glycolide were added and the contents were heated to 135° C. Conditions were maintained until practically complete monomer conversion was achieved. The magnetic stir bar was removed and replaced with a stainless steel mechanical stirrer. The polymer was cooled to room temperature. For a third charge, 1,6-diisocyanatohexane was added and the contents were stirred until complete mixing was achieved. The contents were stirred and heated to 100° C. Conditions were maintained for 1.25 hours. The polymer was allowed to cool to room temperature and then dissolved in an equal part of tetrahydrofuran. The polymer solution was treated with 5 mL of 2-propanol at 55° C. then precipitated in cold water. The purified polymer was dried to a constant weight at 55° C. on a rotary evaporator. The purified polymer was characterized for molecular weight by GPC using tetrahydrofuran as the mobile phase. Identity and composition were confirmed by FT-IR and NMR, respectively.

### Example 5

### Synthesis and Characterization of Typical Polyether-Ester-Urethanes

### Using the General Method of Example 4, P2, P3, and P4

Polyether-ester-urethanes P-2, P-3, and P-4 were prepared using the method of Example 4 with 0.10, 0.225, 0.15 moles of poly(ethylene glycol) (Mₙ=400 Da), 1.73×10⁻⁴, 3.18×10⁻⁴, 2.60×10⁻⁴ moles of tin(II) 2-ethyl hexanoate, 0.35, 0.64, 0.52 moles of dl-lactide, 0.09, 0.16, 0.13 moles of glycolide, and 0.12, 0.18, 0.12 moles of 1,6-diisocyanatohexane, respectively. Polymers P-2, P-3, and P-4 were characterized for molecular weight by GPC using tetrahydrofuran as the mobile phase which resulted in Mₙ of 12, 9, and 9 kDa, M_{w} of 26, 14, and 15 kDa, Mₚ of 22, 12, and 14 kDa, and PDI of 2.1, 1.6, and 1.6, respectively. Identity and composition were confirmed by FT-IR and NMR, respectively.

### Example 6

### Preparation and Evaluation of Bioactive Formulations

### Using Polyurethane Composition P2 from Example 5: General Method

An aliquot of P2 (4.5 g) was heated to 50° C. then mixed thoroughly at that temperature with polyethylene glycol (PEG-400) having a molecular weight of 400 Da (4.4 g). The mixed polymers were allowed to reach room temperature and then thoroughly mixed with a second aliquot of PEG-400 (1.1 g) premixed with the drug solution in ethanol. The final formulation was dried under reduced pressure to distill the ethanol prior to conducting the drug release study. The release profile of the specific drug in the respective formulation was conducted using buffered solution and HPLC as described in Example 4, with the exception of using a buffered saline solution at pH 7.4.

### Example 7

### Preparation and Evaluation of Leflunomide-Containing Formulation F2-A

Following the general method of Example 6, an aliquot of an ethanol stock solution (100 mg/mL) was used to provide a drug concentration in the final formulation of 1.83 weight percent. The drug release results indicated a 0.5, 0.9, and 1.0 percent release at day 1, 3, and 10, respectively.

### Example 8

### Preparation and Evaluation of Paclitaxel-Containing Formulation F2-B

Following the general method of Example 6, an aliquot of an ethanol stock solution (3.33 mg/mL) was used to provide a drug concentration in the final formulation of 0.009 weight percent. The drug release results indicated 1.7 and 1.9 percent release at 1 and 7 days, respectively.

### Example 9

### Synthesis and Characterization of Typical Polyether-Ester-Urethanes

### Using the General Method of Example 4, P-5 to P-8

Polyether-ester-urethanes P-5, P-6, P-7 and P-8 were prepared using the method of Example 4 with 0.15, 0.22, 0.22, 0.22 moles of polyethylene glycol (Mₙ=400 Da), 3.53×10⁻⁴, 4.17×10⁻⁴, 4.22×10⁻⁴, 4.12×10⁻⁴ moles of tin(II) 2-ethyl hexanoate, 0.88, 0.94, 1.08, and 0.80 moles of trimethylene carbonate (TMC), 0.00, 0.31, 0.19, and 0.43 moles of glycolide, and 0.12, 0.18, 0.18, and 0.18 moles of 1,6-diisocyanatohexane, respectively. Polymers P-5, P-6, P-7 and P-8 were characterized for molecular weight by GPC using tetrahydrofuran as the mobile phase which resulted in Mₙ of 11, 10, 10, and 9 kDa, M_{w} of 19, 14, 16, and 14 kDa, Mₚ of 18, 13, 15, and 14 kDa, and PDI of 1.7, 1.4, 1.6 and 1.5, respectively. Identity and composition were confirmed by FT-IR and NMR, respectively.

### Example 10

### Synthesis and Characterization of Acetylated Polyethylene Glycol-400 Da (PG-4A) for Use as a Diluent Liquid Excipient of P-2 to P-8

Predried polyethylene glycol having a molecular weight of about 400 Da (25.6 g) was mixed in a round-bottom flask (equipped for magnetic stirring and refluxing) under dry nitrogen atmosphere with purified acetic anhydride (22.2 g). The mixture was stirred for 1 hour at 40° C. and then at 100° C. for 3 hours. At the conclusion of the reaction, the contents of the flask were heated under reduced pressure to remove the acetic acid reaction by-product and excess acetic anhydride. The acetylated product (PG-4A) was characterized for identity by infrared spectroscopy and molecular weight by gel permeation chromatography (GPC).

### Example 11

### Synthesis, Characterization, and Testing of a Typical Film-Forming Polyether-Urethane-Urea, PEUU-I

Poly(ethylene glycol)-block-poly(propylene glycol)-block-poly(ethylene glycol) (Mₙ=14,600 Da, 82.5 wt % poly(ethylene glycol) (1.64×10⁻³ moles) and poly(tetramethylene glycol) (Mₙ=2,900 Da) (1.93×10⁻² moles) were added to a 500 mL glass resin kettle equipped for mechanical stirring and vacuum. The contents were dried at 140° C. under reduced pressure for 3 hours and then cooled to room temperature. N,N-Dimethylacetamide (190 mL) was added and the contents were heated to 60° C. and stirred until a homogeneous solution was obtained. The contents were cooled to room temperature and 1,6-diisocyanatohexane (3.14×10⁻² moles) was added. The contents were stirred until a homogeneous solution was obtained. Tin(II) 2-ethyl hexanoate (3.53×10⁻⁴ moles) in the form of a 0.2 M solution in 1,4-dioxane was added. The contents were stirred until a homogeneous solution was obtained and then heated to 100° C. under stirring conditions. Conditions were maintained for 2 hours. The contents were cooled to room temperature. Ethylene diamine (1.05×10⁻² moles) was added in the form of a 1.16 M solution in N,N-dimethylacetamide under stirring conditions. Upon gelation, the stirrer was stopped and conditions were maintained for 24 hours. The polymer was purified by subsequent extractions with water and acetone then dried to a constant weight at 45° C. under reduced pressure. The purified polymer was characterized for molecular weight by inherent viscosity in hexafluoroisopropanol which resulted in an inherent viscosity of 5.71 dL/g. Identity was confirmed by FT-IR.

### Example 12

### Preparation and Properties of n-Butyl Cyanoacrylate-Based Tissue Adhesive Formulation

### Using P-5 as a Rheology Modifier

This entailed mixing and characterizing the different monomer combinations and using a selected mixture to prepare a typical adhesive formulation.

A pure methoxypropyl cyanoacrylate (MPC) and pure n-butyl cyanoacrylate (BC) monomers and combination thereof were characterized for their rheological properties, measured in terms of their comparative viscosity as listed Table II. Ratios of 90/10, 50/50, 20/80, and 10/90 (by weight) of MPC to butyl cyanoacrylate were mixed. Monomers were weighed in a centrifuge tube and placed on a shaker for 15 minutes. The rheological data of the resulting compositions are summarized in described in Table II.

TABLE II
Cyanoacrylate Monomer Compositions and Their Rheological Data

| Monomer Ratios | Monomer Compositions | Comparative Viscosity (s) |
|---|---|---|
| 100 | BC | 3.30 ± 0.06 |
| 10:90 | MPC:BC | 3.42 ± 0.06 |
| 20:80 | MPC:BC | 3.55 ± 0.10 |
| 50:50 | MPC:BC | 4.23 ± 0.14 |
| 90:10 | MPC:BC | 5.16 ± 0.17 |
| 100 | MPC | 6.15 ± 0.36 |

^{a}Measured in terms of time (in seconds) to collect 0.3 mL of liquid adhesive, transferring vertically by gravity through an 18-guage, 1.5 in. long syringe needle.

A selected formulation was prepared by dissolving 3% (by weight) of P1 in a 20/80 (by weight) mixture of methoxypropyl cyanoacrylate and butyl cyanoacrylate containing 500 ppm of butylated hydroxyanisole and 3.3 ppm of pyrophosphoric acid stabilizers against free radical and anionic polymerization, respectively. More specifically, this entailed the following steps: (1) the P1 polymer was added to a flask and dried at 80° C. for 3 hours; (2) the cyanoacrylate monomers and the stabilizers were added; and (3) the resulting mixture was stirred at 80° C. until it became homogenous. The resulting formulation exhibited a comparative adhesive viscosity of 12.63 s and an adhesive joint strength of 28.35 N (using a fabric peel test).

### Example 13

### Preparation and Properties of Absorbable Cyanoacrylate Tissue Adhesive Formulation

### Using P-6 as a Rheology Modifier

The adhesive formulation was prepared by dissolving 5% (by weight) of P-6 in a 90/10 (by weight) mixture of methoxypropyl cyanoacrylate and ethyl cyanoacrylate containing 500 ppm of butylated hydroxyanisole and 3.3 ppm of pyrophosphoric acid as stabilizers against free radical and anionic polymerization, respectively. More specifically, this entailed the following steps: (1) the P3 polymer was added to a flask and dried at 80° C. for 3 hours; (2) the cyanoacrylate monomers and stabilizers were added; and (3) the resulting mixture was stirred at 80° C. until it became homogenous. The resulting formulation exhibited a comparative adhesive viscosity of 6.74 s and an adhesive joint strength of 34.96 N (using a fabric peel test).

### Example 14

### Preparation of a Doxycycline Hyclate Controlled Release Formulation

### Using P-2 and Determination of the Drug Release Profile

This entailed a three-step process, namely, mixing P-2 (from Example 5) with a diluent liquid excipient (from Example 10), acetylated polyethylene glycol-400 (PG-4A), preparation of an active formulation, and monitoring the drug release profile.

Mixing P-2 with PG-4A

For this, P-2 (3.2691 g) was placed in a glass vial and PG-4A (1.7603 g) was added. The contents of the vial were heated to 50° C. and mechanically mixed until a homogenous mixture developed. The final mixture was 65 weight percent P-2 with the remainder consisting of PG-4A.

Preparation of Active Formulation

To prepare a liquid vehicle, an aliquot of 2.0237 g of the P-2/PG-4A mixture was transferred to a glass vial, and doxycycline hyclate (434 mg) was added to the vial. Microparticles of acid-terminated polyglycolide (433 mg) were added to the contents of the vials. This was followed by heating to 50° C. and mixing mechanically to obtain a homogenous mixture. The resulting mixture was 70 weight percent liquid vehicle, 15 percent polyglycolide microparticles, and 15 percent doxycycline hyclate.

Release Study

The active formulation (1.0230 g) was placed in a small glass vial and heated to 50° C. to flow into bottom of vial and create a uniform coating and then was allowed to cool to room temperature. Phosphate buffer (10 mL, pH 7.2) was placed into the glass vial, which was transferred to a 37° C. incubator. The buffered solution (with released drug) was withdrawn at predetermined time points and replaced with 10 mL of fresh buffer. Aliquots of the release buffer were assayed by reverse phase HPLC, using a Waters Chromatography System with a C18 column, a gradient of 15-30% acetonitrile over 10 minutes, and detection at 350 nm; the amount of doxycycline released over time was determined. The HPCL data indicated a cumulative release at 23, 94, and 163 hours of 16%, 31%, and 45%, respectively.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the following claims. Moreover, Applicant hereby discloses all subranges of all ranges disclosed herein. These subranges are also useful in carrying out the present invention.

What is claimed is:

1. A method for delivering an active agent, the method comprising:
a) providing a liquid injectable composition comprising an active agent, a liquid polyether, and an absorbable segmented aliphatic polyurethane;
b) injecting the composition into a biological site; and
c) releasing the active agent from the composition and into the biological site.

2. The method of claim 1 wherein the composition does not contain organic solvent.

3. The method of claim 1 wherein the polyether is a liquid polyoxyalkylene comprising chains of at least one type of oxyalkylene sequence selected from oxyethylene, oxypropylene and oxytrimethylene.

4. The method of claim 1 wherein the liquid polyether is water soluble.

5. The method of claim 1 wherein the polyurethane comprises polyoxyalkylene chains covalently linked to alkylene carbonate chains.

6. The method of claim 1 wherein the polyurethane is prepared by end-grafting polyethylene glycol having a molecular weight of about 400 Da with trimethylene carbonate, and interlinking the end-grafted polyethylene glycol with hexamethylene diisocyanate.

7. The method of claim 1 wherein the polyurethane comprises polyether and polyester segments.

8. The method of claim 1 wherein the polyurethane comprises polyether-carbonate-urethane-urea.

9. The method of claim 1 wherein the polyurethane comprises polyether-carbonate-urethane.

10. The method of claim 1 wherein the polyurethane comprises polyether-carbonate-ester-urethane.

11. The method of claim 1 wherein the polyurethane comprises polyether-ester-urethane.

12. The method of claim 1 wherein the active agent is an antineoplastic agent.

13. The method of claim 1 for treating cancer.

14. The method of claim 1 wherein the active agent is an antimicrobial agent.

15. The method of claim 1 wherein the active agent is an immunosuppressant agent.

16. The method of claim 1 wherein the active agent is an antifungal agent.

17. The method of claim 1 wherein the active agent is an antibacterial agent.

18. The method of claim 1 wherein the active agent is a cross-over bioactive agent.

19. The method of claim 1 wherein the injection is by syringe.

20. The method of claim 1 wherein the injection is by collapsible dispenser.
